(19) United States
(12) Patent Application Publication
(10) Pub. No.: US 2015/0305876 A1
(43) Pub. Date: Oct. 29, 2015

(54) IMPLANTABLE GLENOID PROSTHESES

(71) Applicant: Topsfield Medical GmbH, Berlin (DE)

(72) Inventors: J. Christopher Flaherty, Auburndale, FL (US); Charnice Barbour, Cambridge, MA (US); Ross Connor, Henniker, NH (US); Christopher Zentner, North Seattle, WA (US); R. Maxwell Flaherty, Auburndale, FL (US)

(21) Appl. No.: 14/750,118

(22) Filed: Jun. 25, 2015

Related U.S. Application Data
(63) Continuation of application No. 14/005,061, filed on Jan. 23, 2014.
(60) Provisional application No. 61/452,236, filed on Mar. 14, 2011.

(51) Int. Cl. A61F 2/40; A61B 17/86; A61B 17/84; A61F 2/30; A61F 2/46

(52) U.S. Cl. CPC A61F 2/4081; A61F 2/30771; A61F 2/46; A61B 17/842; A61B 17/86; A61F 2/30; A61F 2002/30029; A61F 2002/30098; A61F 2002/30772; A61F 2002/30878; A61F 2002/3082; A61F 2002/4631; A61F 2310/00023

(57) ABSTRACT

An implantable glenoid prosthesis comprising a glenoid member including a glenoid body and a glenoid fixation member is disclosed. The glenoid body includes a surface for mating with a humeral head. The glenoid fixation member is constructed and arranged to flex when a force is applied to the glenoid body.

### RELATED APPLICATIONS

This application is a continuation application of U.S. patent application Ser. No. 14/005,061, filed on Sep. 13, 2013 which claims the benefit of International Application Serial No. PCT/US2012029045, filed Mar. 14, 2012, which claims the benefit of U.S. Provisional Application Ser. No. 61/452,236, filed Mar. 14, 2011, the content of which is incorporated herein by reference, in its entirety.

### FIELD OF THE APPLICATION

Embodiments of the present application relate generally to implantable prostheses, and more particularly, to implantable glenoid prostheses that include one or more flexible portions, and methods of implanting prostheses.

### BACKGROUND

Many joints of the human body naturally articulate relative to one another. Generally, the articulation surfaces of these joints are substantially smooth and without abrasion. However, joints, such as shoulder joints, undergo degenerative changes due to a variety of causes, such as, disease, injury and various other issues. When these degenerative changes become advanced, to the point of becoming irreversible, such joints or portions thereof may need to be replaced with one or more prosthetics.

In light of the degenerative changes found in shoulder joints, various shoulder prosthetics of conventional design have been proposed. However, conventional shoulder prosthetics and their associated surgical components suffer from many disadvantages. For example, glenoid components of conventional design are subject to various types of load forces, such as, shear forces, anterior/posterior forces, lateral/medial forces, and rotational forces, which may cause notching and chipping of bone and/or loosening of components, thereby reducing the lifespan of the prosthetic. In addition, such load forces may create a rocking moment causing glenoid components to lift, which can further result in notching and chipping of bone and/or separation of the glenoid component from a scapula. Furthermore, the loosening of conventional shoulder prosthetics may pulverize, grind, crush and deform portions of a scapula, for example, a glenoid cavity of a scapula, which as a result can prohibit the replacement of a worn, damaged or non-functional shoulder prosthetic. For these and other reasons, there is a need for improved shoulder prosthetics.

### SUMMARY

Embodiments of the present application are directed toward implantable glenoid prostheses, methods of implanting glenoid prostheses and surgical tools for implanting glenoid prostheses that further address and reduce notching and chipping of bone and component loosening associated with implantable glenoid prosthetics. In particular, embodiments provide implantable glenoid prostheses and methods of implantation that realize, among other features, a flexing characteristic that reduces an applied load force through the absorption and dissipation of said force, and avoidance of forces being created between the glenoid prosthesis and the scapula in which it has been implanted. Although embodiments may be described with reference to glenoid prosthesis, joint components and methods for implantation described herein are applicable to other joints, such as hips, knees, elbows, wrists, digits and other joints. Patients applicable to these prosthetics include humans and other mammals, as well as other animalia.

According to one aspect, an implantable glenoid prosthesis comprises a glenoid body comprising a glenoid joint surface configured to provide a bearing surface for a head portion of a humerus and a glenoid fixation member configured to attach the glenoid body to a scapula. The glenoid fixation member is further configured to flex when a force is applied to the glenoid body.

In various embodiments, the glenoid prosthesis can include one or more glenoid fixation members. The glenoid fixation member can approximate the flexibility of the scapula, or can be more flexible than the scapula. The glenoid fixation member is configured to bend in unison with the scapula thus reducing the magnitude of opposing movements and/or forces. The glenoid fixation member is configured to flex in at least one of the following ways: axial flexing; radial flexing or torsional flexing, and in some embodiments, the glenoid fixation member is configured to flex in at least two of these ways. The glenoid fixation member is configured to reduce one or more forces transmitted to the scapula when a force is applied to the glenoid fixation member and/or reduce one or more forces transmitted to the glenoid body when a force is applied to the scapula.

In various embodiments, the glenoid fixation member can comprise a shaped memory alloy material such as Nitinol, configured to undergo a phase change. The phase change can occur when the shaped memory alloy material is heated and/or cooled, for example, heated to body temperature. The shaped memory alloy material can be configured to pivot at least a portion of the glenoid fixation member. For example, the glenoid fixation member can comprise a peg having a proximal portion and a distal portion connected via a joint where a shaped memory alloy wire undergoes a phase change causing the peg distal portion to hinge at the joint. In some cases, the wire undergoes approximately a 6% to 8% strain during the phase change. The shaped memory alloy material can comprise a foldable flange. The shaped memory alloy material can comprise a tube having multiple slits along a portion of its length. The glenoid fixation member can further comprise an implantable tube, where the shaped memory alloy material is configured to extend beyond and engage the tube when implanted.

In various embodiments, the glenoid fixation member can comprise a linear or a non-linear geometry. In some embodiments, the prosthesis further comprises a second fixation member wherein the glenoid fixation member and the second glenoid fixation member comprise a non-linear geometry, for example a helical geometry.

In various embodiments, the glenoid fixation member can comprise a material selected from the group of materials consisting of: cobalt-chrome; titanium; stainless steel; tantalum; polyethylene; Delrin; silicon; nylon; and combinations of these. The glenoid fixation member can further comprise a shaped memory alloy material such as Nitinol. The shaped memory alloy can undergo a phase change such that a retention force between the scapula and glenoid fixation member is increased.

In various embodiments, the glenoid fixation member can be configured to be inserted into a hole, for example a hole having a diameter approximating 0.04″. The hole can further comprise a radially extended distal portion.

In various embodiments, the glenoid fixation member can extend into the scapula in a medial direction.

In various embodiments, the glenoid fixation member can include at least one rigid portion and/or at least one flexible portion.

In various embodiments, the glenoid fixation member can be selected from the group consisting of: a fin, a pin, a peg and a screw. The glenoid fixation member can include a keel construction. The glenoid fixation member can include a wire construction, for example a wire construction including multiple wires. The wire(s) can comprise varying geometries, for example straight, curved, or helically shaped. The glenoid fixation member can include at least one in-growth element, for example an in-growth element selected from the group consisting of: a hole; a projection; a flange; a notch; a recess; a groove; and combinations of these.

In various embodiments, the head portion of the humerus can be a prosthetic implant portion.

In various embodiments, the glenoid body can comprise a material selected from the group of materials consisting of: cobalt-chrome; titanium; stainless steel; tantalum; polyethylene; Delrin; silicon; nylon; and combinations of these. The glenoid body can further comprise a shaped memory alloy material such as Nitinol.

In various embodiments, the glenoid joint surface can surround a humeral joint surface such that movement of a humeral bone is at least partially constrained in two directions. The glenoid joint surface can be concave where a mating humeral joint surface is convex. Conversely, the glenoid joint surface can be convex where a mating humeral joint surface is concave.

In various embodiments, the glenoid prosthesis can further comprise bone cement.

According to another aspect, a method for implanting a glenoid prosthesis comprises implanting a glenoid body and attaching the glenoid body to a scapula via a glenoid fixation member where the glenoid fixation member is configured to flex when a force is applied to the glenoid body.

In various embodiments, the glenoid fixation member can comprise a shaped memory alloy material such that the attachment of the glenoid body to the scapula occurs upon a phase change of the shaped memory alloy material. The phase change can occur upon heating and/or cooling of the shaped memory material, for example upon a transition to body temperature. Alternatively or additionally, the shaped memory alloy material can be heated by passing a current through the material or via a heating device such as a heat gun. In some embodiments, the phase change causes the shaped memory alloy material to pivot at least a portion of the glenoid fixation member. In some embodiments, the phase change causes the shaped memory alloy material to mechanically engage the scapula.

In various embodiments, the glenoid fixation member can comprise at least one in-growth element selected from the group consisting of: a hole; a projection; a flange; a notch; a recess; a groove; and combinations of these, where the glenoid body attaches to the scapula via bone in-growth.

In various embodiments, the glenoid fixation member can comprise a retaining tube having a proximal end and a distal end, and at least one wire where the at least one wire is advanced such that it engages the distal end of the retaining tube. The wire can comprise a shaped memory alloy material constructed and arranged to undergo a phase change such that the phase change causes the wire to advance and engage the retaining tube.

In various embodiments, the glenoid fixation member can comprise a flange where the glenoid body is attached to the scapula via folding the flange. In some embodiments, the flange can be manually folded. Alternatively, the flange can comprise a shaped memory alloy material where the flange is folded via a phase change of the shaped memory alloy material.

In various embodiments, the glenoid body can be attached to the scapula via at least one of: bone cement; at least one bone screw; or a press fit.

In various embodiments, the method can further comprise drilling at least one hole in the scapula. The at least one hole can comprise a diameter of approximately 0.04″. The at least one hole can further comprise a radially extended distal portion.

In various embodiments, the method can further comprise securing the glenoid fixation member to the glenoid body. In one embodiment, the glenoid fixation member comprises a proximal end and a distal end, and the proximal end is secured to the glenoid body via at least one of: a weld; a crimp; or an adhesive joint.

In various embodiments, the method can further comprise reversing or loosening the attachment of the glenoid body to the scapula. For example, where the glenoid fixation member comprises shaped memory alloy material, the material can be cooled. A cooled saline solution or a heat removal device can be used to cool the shaped memory alloy material.

### BRIEF DESCRIPTION OF THE DRAWINGS

The foregoing and other objects, features and advantages will be apparent from the more particular description of preferred embodiments, as illustrated in the accompanying drawings in which like reference characters refer to the same parts throughout the different views. The drawings are not necessarily to scale, emphasis instead being placed upon illustrating the principles of the preferred embodiments.

FIG. 1A is an anterior facing environmental view of a left shoulder joint;

FIG. 1B is a posterior facing environmental view of a left shoulder joint;

FIG. 1C is a lateral/medial facing view of a scapula;

FIGS. 2A and 2B are side and end views, respectively, of an implantable glenoid prosthesis including a flexible keel-type fixation member, consistent with the present inventive concepts;

FIG. 3A is a side view of an implantable glenoid prosthesis including flexible, shaped memory fixation wires, consistent with the present inventive concepts;

FIG. 3B is a side view of the implantable glenoid prosthesis of FIG. 3A with the shaped memory wires transitioned to a curved state, consistent with the present inventive concepts;

FIGS. 4A through 4H are side views of various configurations of shaped memory fixation wires, shown after a phase transition, consistent with the present inventive concepts;

FIG. 5A is a side view of an implantable glenoid prosthesis including two fixation wires and a fixation peg, consistent with the present inventive concepts;

FIG. 5B is a side view of an implantable glenoid prosthesis including two fixation wires and three fixation pegs, consistent with the present inventive concepts;

FIGS. 6A and 6B are side views of two different implantable glenoid prostheses, consistent with the present inventive concepts;

FIGS. 6C through 6F are surface views of four different implantable glenoid prosthesis with various surface wire patterns, consistent with the present inventive concepts;

FIGS. 6G through 6I are end views of three different wire fixation cross sectional profiles, consistent with the present inventive concepts;

FIGS. 7A and 7B are side views of a pre-deployed and post-deployed, respectively, wire fixation member, consistent with the present inventive concepts;

FIG. 8 is a side view of an implantable glenoid prosthesis including a cork-screw fixation member, consistent with the present inventive concepts;

FIG. 9 is a side view of an implantable glenoid prosthesis including two split end fixation wires, consistent with the present inventive concepts;

FIGS. 10A and 10B are side and end views, respectively, of a tubular fixation element with radially extending portions, consistent with the present inventive concepts;

FIGS. 10C and 10D are side and end views, respectively, of the tubular fixation element of FIGS. 10A and 10B with the radially extending portions deployed, consistent with the present inventive concepts;

FIG. 10E is a side view of an implantable glenoid prosthesis including the tubular fixation element of FIGS. 10A through 10D, shown in the deployed condition, consistent with the present inventive concepts;

FIGS. 11A and 11B are surface views of an undeployed and deployed, respectively, implantable glenoid prosthesis including a flange surface which wraps from the glenoid surface to the side of the scapula, consistent with the present inventive concepts;

FIGS. 12A and 12B are side views of an undeployed and deployed, respectively, implantable glenoid prosthesis including peg fixation members which include a deployable distal portion, consistent with the present inventive concepts; FIG. 12C is a surface view of the implantable glenoid prosthesis of FIGS. 12A and 12B.

### DETAILED DESCRIPTION OF VARIOUS EMBODIMENTS

Reference will now be made in detail to the present embodiments, examples of which are illustrated in the accompanying drawings. Wherever possible, the same reference numbers will be used throughout the drawings to refer to the same or like parts.

Applicant's copending international application, Ser. No. PCT/US11/38096, titled “IMPLANTABLE PROSTHESES”, filed on May 26, 2011, is incorporated by reference herein in its entirety.

The implantable glenoid prosthesis of the present inventive concepts includes a glenoid member attached to one or more fixation members configured to attach the glenoid member to the scapula of a patient. The fixation members are constructed and arranged to allow flexing or twisting after implantation. The fixation members are placed into a scapula of a patient, typically in one or more holes, notches or other recess made during implantation surgery. In some embodiments, the fixation members are constructed of a shaped memory alloy, or include portions made of a shaped memory alloy, such as Nitinol.

The use of elastic materials such as Nitinol for glenoid fixation, similar to Nitinol screws and some applications of cement, supports bone in-growth on another part of the implant (e.g. a hole or partial recess) by resisting initial micromotion or other small movements that may occur while forces are applied to the implant or the patient's scapula. At the same time, glenoid fixation members with thin cross-sections and/or small diameters can minimize bone resection (e.g. slots can be made or smaller holes can be drilled, e.g. approximately 0.04″ or smaller). This minimal removal of bone material makes revision surgery more viable because there is more bone available, should the glenoid fixation member have to be removed. The shaped memory aspect of the glenoid fixation member can be used to cause immediate fixation, shortening surgery time, lowering blood loss and speeding up recovery.

One of the many disadvantages to the screw and cement approaches commonly used today, is the eccentric loading of the glenoid, known as the “rocking-horse” motion. This “rocking-horse” motion is a problem for fixation solutions including projections held in place with bone cement. The rigidity that results after implantation unduly resists motion, which can cause loosening. The glenoid fixation members of the present inventive concepts provide sufficient initial stabilization for bone in-growth to occur. Fixation increases over time and results in a more natural support by the glenoid component. Use of screws for glenoid fixation often requires a metal backed glenoid component. This configuration introduces issues with modular implants such as dissociation between the articulating implant and the metal backing, backside wear of the articulating piece, stress shielding (the forces from normal joint movement are not naturally transferred to a scapula, which prevents proper bone recovery), and joint overstuffing.

Using a glenoid fixation member (e.g. a Nitinol or other elastic material) molded into a polycarbonate-urethane component avoids the requirement of a metal-backed implant, while providing opportunities for bone in-growth and resistance to micromotion disturbances. A Nitinol material design provides the level of fixation required to allow bone in-growth (e.g. by using phase change characteristics to achieve immediate fixation), and will be able to better handle eccentric loading because of its super-elastic nature. Also, Nitinol has a similar stress-strain profile to natural bone. This characteristic allows the fixation member to “share” and transfer loads evenly with the cancellous bone of a scapula. This mimicking of the native bone characteristic will encourage the shoulder to heal faster and stronger, and reduces the problem of stress shielding.

In one embodiment, a Nitinol based fixation member may be used with a polycarbonate-urethane polymer for the glenoid body. Both the Nitinol and the polycarbonate-urethane are biocompatible, and the polycarbonate-urethane is much more compliant than UHMWPE, providing a bearing surface that may more closely mimics the soft tissue of the shoulder. This arrangement will be better suited for shock absorption, such as from eccentric loading, and act as a dampener for less traumatic distribution of load as the humeral head contacts the scapula. Nitinol can also be visualized in the body using magnetic resonance imaging, allowing for better visualization of fixation and easier patient follow-up.

Embodiments are described more fully hereinafter with reference to the accompanying drawings, in which exemplary embodiments are shown. The present inventive concepts may, however, be embodied in many different forms and should not be construed as limited to the example embodiments set forth herein. Rather, these exemplary embodiments are provided so that this disclosure will be thorough and complete. In the drawings, the sizes and relative sizes of objects may be exaggerated for clarity.

It will be understood that when an element or object is referred to as being “on,” “connected to” or “coupled to” another element or object, it can be directly on, connected or coupled to the other element or object, or intervening elements or objects may be present. In contrast, when an element is referred to as being “directly on,” “directly connected to” or “directly coupled to” another element or object, there are no intervening elements or objects present. As used herein, the term “and/or” includes any and all combinations of one or more of the associated listed items.

It will be understood that, although the terms first, second, etc. are used herein to describe various elements, these elements should not be limited by these terms. These terms are used to distinguish one element from another. For example, a first element could be termed a second element, and, similarly, a second element could be termed a first element, without departing from the scope of the present inventive concepts. As used herein, the term “and/or” includes any and all combinations of one or more of the associated listed items.

The terminology used herein is for the purpose of describing particular embodiments only and is not intended to be limiting of the present inventive concepts. As used herein, the singular forms “a,” “an” and “the” are intended to include the plural forms as well, unless the context clearly indicates otherwise. It will be further understood that the terms “comprises” and/or “comprising,” when used in this specification, specifically the presence of stated features, integers, steps, operations, elements, and/or components, but do not preclude the presence or addition of one or more other features, integers, steps, operations, elements, components, and/or groups thereof.

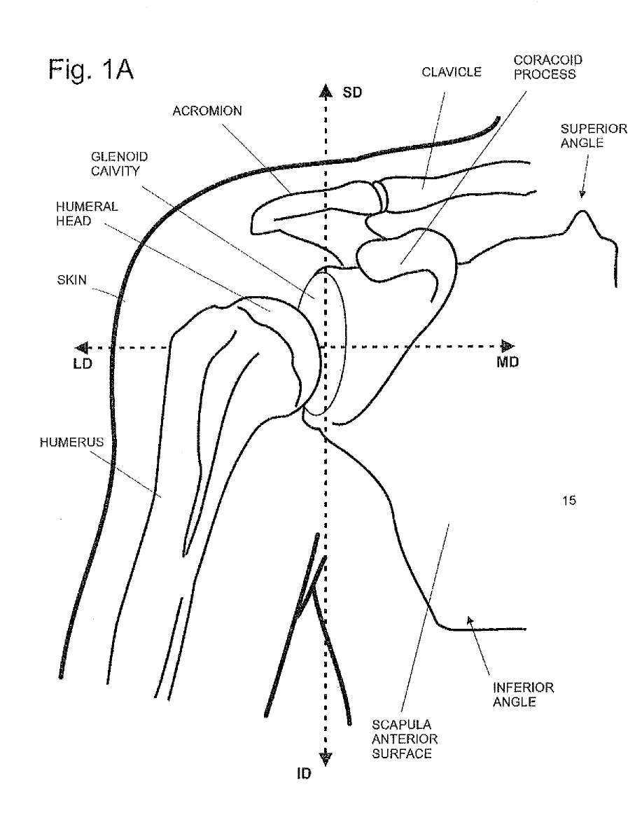

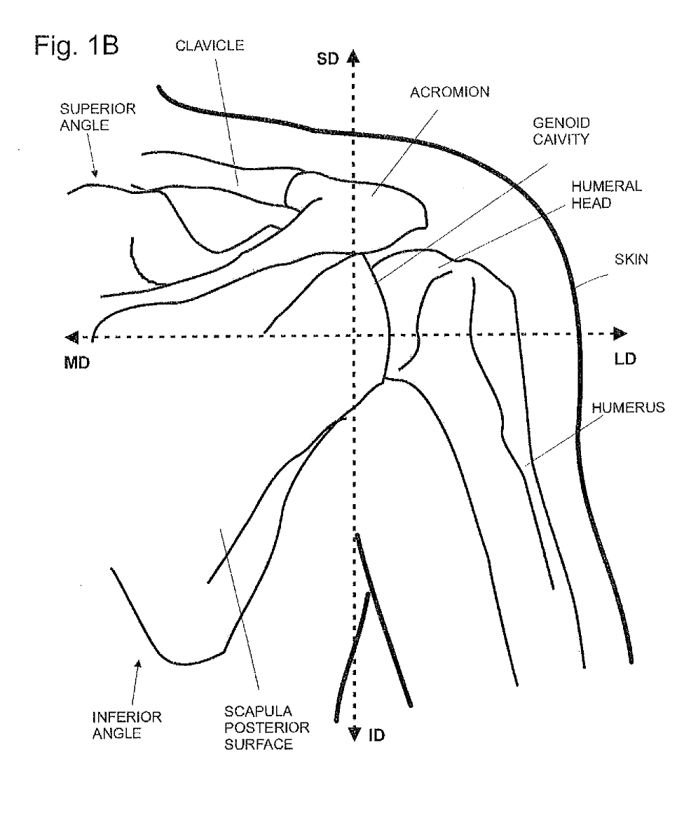

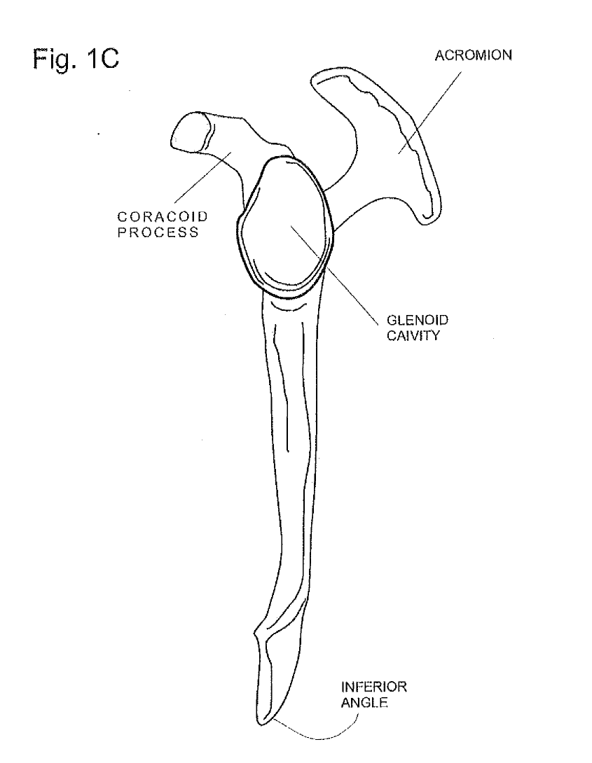

FIG. 1A is an anterior facing environmental view of a shoulder joint, FIG. 1B is a posterior facing environmental view of a shoulder joint, and FIG. 1C is a lateral/medial facing view of a scapula. In human anatomy, a shoulder joint comprises the part of the body where a humeral bone (i.e., humerus) attaches to a shoulder blade (i.e., scapula). The humerus comprises a humeral head portion that interfaces with a glenoid cavity of a scapula, such that the humerus articulates with respect to the glenoid cavity of a scapula. A scapula forms the posterior located part of the shoulder girdle.

For purposes of the present disclosure, the terms “sagittal plane” and the like, when referring to portions of the human body, refers to an imaginary plane that travels vertically from the top to the bottom of the body, dividing the body into left and right portions.

For purposes of the present disclosure, the terms “coronal plane”, “frontal plane” and the like, when referring to portions of the human body, refers to an imaginary plane that travels vertically from the top to the bottom of the body, dividing the body into anterior and posterior (e.g., belly and back) portions.

For purposes of the present disclosure, the terms “medial”, “medial direction” and the like, when referring to anatomical terms of direction, refers to a direction that is transverse to the sagittal plane of a human body, and that extends in a direction toward the sagittal plane of a human body.

For purposes of the present disclosure, the terms “lateral”, “lateral direction” and the like, when referring to anatomical terms of direction, refers to a direction that is transverse to the sagittal plane of a human body, and that extends in a direction away from the sagittal plane of a human body.

For purposes of the present disclosure, the terms “superior/inferior”, “superior/inferior direction” and the like, when referring to anatomical terms of direction, refers to a direction that extends in upward and downward directions, through a superior angle of a scapula and an inferior angle of a scapula.

For purposes of the present disclosure, the terms “superior”, “superior direction” and the like, when referring to anatomical terms of direction, refers to a direction that extends upward, through a superior angle of a scapula.

For purposes of the present disclosure, the terms “inferior”, “inferior direction” and the like, when referring to anatomical terms of direction, refers to a direction that extends downward, through an inferior angle of a scapula.

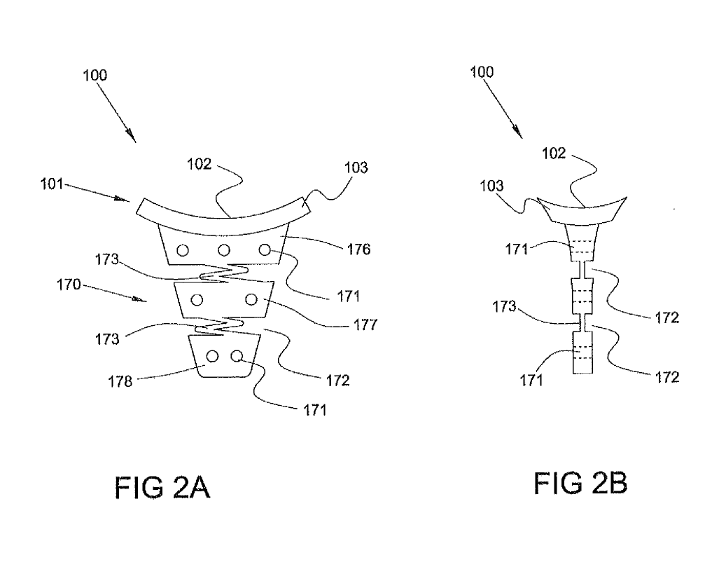

FIGS. 2A and 2B are side and end views, respectively of an implantable glenoid prosthesis including a glenoid body and a flexible glenoid fixation member. Glenoid prosthesis 100 comprises a glenoid member 101 including glenoid joint surface 102 and glenoid body 103. Extending from glenoid member 102 is a glenoid fixation member, fin 170. Fin 170 is preferably manufactured of a flexible or semi-rigid material, such as to achieve a construction with similar properties of the scapula into which fin 170 is to be implanted. Fin 170 may be made of a metal or combination of metals, and have a thickness such that fin 170 can flex under normal load conditions. In one embodiment, fin 170 is made of Nitinol or includes one or more Nitinol portions. In another embodiment, fin 170 is made of a different metal or other material and sized to flex under normal load conditions, such as a material selected from the group consisting of: Nitinol; cobalt-chrome; titanium; stainless steel; tantalum; polyethylene; Delrin; silicon; nylon; and combinations of these.

Glenoid body 103 may be constructed of a metal, plastic or other biocompatible material or materials. In one embodiment, glenoid body 103 is constructed with a polycarbonate-urethane polymer and configured to absorb or otherwise dampen one or more loads applied to glenoid body 103 such as by a natural or artificial humeral head.

Fin 170 comprises a fin-like projection, similar to the keel on a sailing vessel. Fin 170 includes one or more spring portions along its length, such as spring portions 173 shown in FIGS. 2A and 2B. Spring portions 173 include a zig-zag design configured to allow axial compression and extension of, and absorb axial loads upon fin 170. Spring portions 173 are also thinner than the remaining keel portion of fin 170, such as to allow twisting and absorption of torsional loads. Spring portions 173 and the remaining portions of fin 170 are further configured to allow bending along one or more axes in the plane of the keel surface of fin 170. The flex design and construction of fin 170 prevents eccentric and other loading between fin 170 and the scapula into which fin 170 is implanted. The reduction of loads reduces the likelihood of loosening of glenoid prosthesis 100.

Fin 170 may further comprise one or more holes, grooves, partial recesses or other geometric configurations design to allow in-growth of scapular bone into a surface portion of fin 170. Referring back to FIGS. 2A and 2B, fin 170 includes multiple holes 171 which pass from one surface of fin 170 to the opposing surface and are configured to anchor fin 170 into a scapula over time. Fin 170 further includes recesses 172 which also promote stabilization via bone in-growth.

Fin 170 may be placed at the time of surgery using bone cement, one or more bone screws (not shown but typically placed through a thru-hole in the surface of glenoid body 103), or may be temporarily secured via a press fit. In one embodiment, fin 170 may comprise a shaped memory material such as Nitinol, and after placement into a notch made in the patient's scapula, a phase change is initiated changing the shape of one or more portions of fin 170, further securing fin 170 into the notch (e.g. the phase change increases the frictional engagement). One or more portions of fin 170 may comprise a shaped memory material configured to engage the scapula during implantation surgery, such as a component which bends or twists into a surface within the scapula such as a surface of a hole or a notch as it transitions to body temperature. The shaped memory portion may apply a securing force and potentially partially deform a portion of the scapula such as to create a mechanical engagement similar to a screw thread.

Fin 170 comprises a proximal portion 176, a mid portion 177 and a distal portion 178. Flexing in multiple degrees of freedom can be achieved between the three portions. In one embodiment, as proximal portion 176 and/or mid portion 177 become loosened within the scapula over time, distal portion 178 remains secured. Fin 170 is constructed and arranged, while supporting necessary flexion, to have sufficient rigidity to provide support to glenoid body 103 and glenoid surface 102 such that securement by distal portion 178 alone is adequate for clinical efficacious stability of glenoid prosthesis 100.

In one embodiment, fin 170 may be constructed and sized to approximate the material properties of the scapula, such as to approximate the flexibility of the scapula. Alternatively, fin 170 may be constructed and sized to be more flexible than the scapula. Fin 170 may be constructed and arranged to move in unison with the scapula, such as when one or more loads are applied to glenoid joint surface 102 by a humeral head. The flexing properties of fin 170 may be configured to reduce the magnitude of opposing forces and/or movements between fin 170 and the scapula. Fin 170 may be constructed and arranged to flex in multiple directions, such as in an axial direction (along an axis into the scapula) and/or in a radial direction (about an axis in the plane of the scapula). Fin 170 may be constructed and arranged to support torsional flexing. These and other flex characteristics of fin 170 may result in a reduction of forces transmitted from the scapula to fin 170 and/or from fin 170 to the scapula. This reduction in forces will tend to prevent loosening of fixation member 170, prolonging its effective implant life.

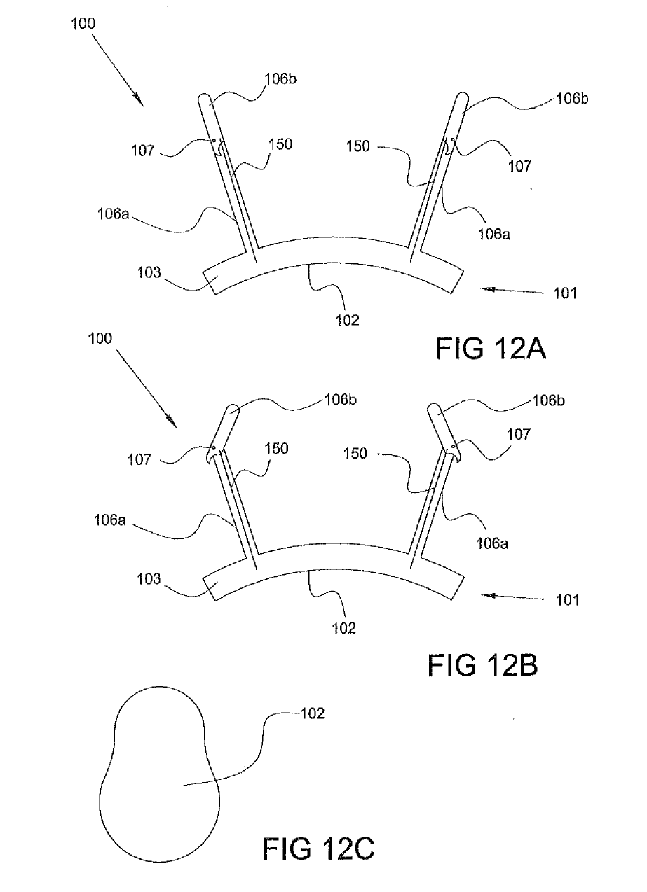

FIGS. 12A and 12B are side views of an implantable glenoid prosthesis in an undeployed and deployed condition, respectively, including a plurality of glenoid fixation members with a distal portion pivotable via a shape memory alloy wire phase transition. Glenoid prosthesis 100 includes at least one glenoid fixation member, a peg having distal portion 106b and proximal portion 106a where portions 106a and 106b are connected via joint 107. Peg proximal portion 106a is attached to glenoid joint surface 102 of glenoid member 101. Glenoid prosthesis 100 also includes shape memory alloy wire 150. Wire 150, typically a Nitinol wire, is configured to undergo a phase transformation that shortens wire 150 and causes peg distal portion 106b to hinge at pivot 107 and further engage with a scapula, as shown in FIG. 12B. One way or two way shaped memory alloys, well known to those of skill in the art, may be used, Peg distal portion 106b may travel into a pre-made pocket within the scapula, may deform and move into a deformed portion of the scapula and/or or may simply apply an increased securing force to the scapula. Generally, a 6-8% strain can be achieved in the phase transition. The transformation is typically initiated by heating wire 150, such as by passing current through wire 150 (power supply and connections not shown), or by heating with a heating device such as a heat gun. Alternatively or additionally, wire 150 may be heated by the patient's body temperature, such as when wire 150 is maintained at room temperature or below prior to implantation in the patient. Cooling, such as cooling using cooled saline or a heat removing device can be used to change the shape of wire 150 and/or to reverse the changes that occurred during heating. Various one way and two way shape memory alloys having various transformation temperatures may be utilized in glenoid prosthesis 100.

FIG. 12C is a surface view of glenoid joint surface 102 which is the mating surface of glenoid member 101 with the humeral head. In one embodiment, the glenoid joint surface 102 is concave, such that the glenoid joint surface 102 is constructed and arranged to interface with a convex humeral joint surface of a head portion of a humeral member. In another embodiment, the glenoid joint surface 102 of the glenoid member 101 is convex, such that, the glenoid joint surface 102 is constructed and arranged to interface with a concave humeral joint surface of a head portion of a humeral member. (e.g., reverse shoulder prosthetic). In these embodiments, the humeral member can comprise a humeral bone of a human being or an artificial humeral prosthetic, or combinations of these.

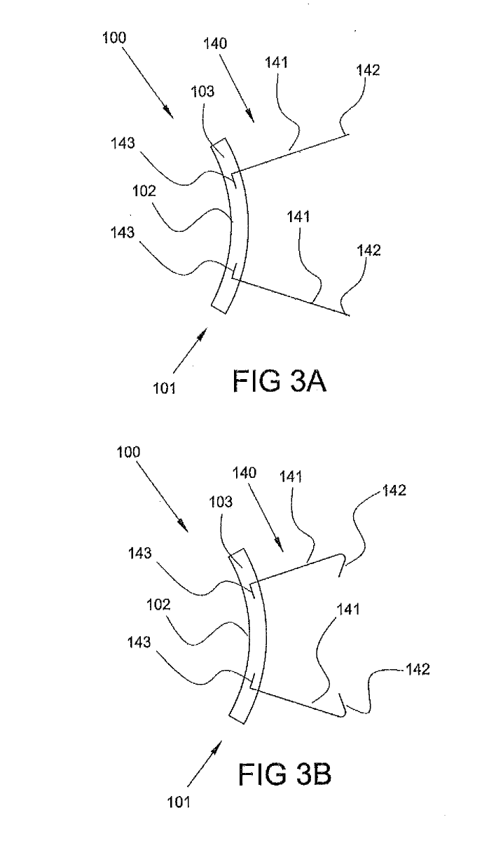

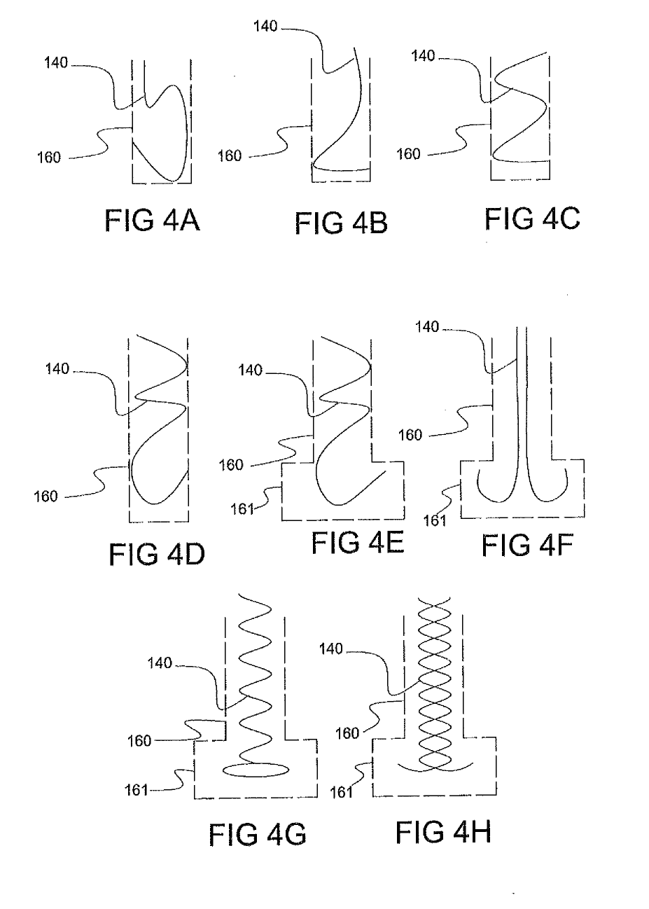

FIGS. 3A and 3B are side views of an implantable glenoid prosthesis, shown in undeployed and deployed conditions, respectively, and including a plurality of shape memory alloy fixation wires. Glenoid prosthesis 100 includes glenoid member 101 having glenoid body 103 with glenoid surface 102. Glenoid prosthesis 100 further includes at least one fixation element, wire 140 having distal end 142, proximal end 143, and body portion 141 therebetween. Proximal end 143 is secured to glenoid body 103, such as via a weld, a crimp and/or an adhesive joint. Fixation wire 140 is preferably a shape memory alloy wire, for example, a Nitinol wire configured to undergo a phase change transformation, such as a phase change transformation that occurs when wire 140 is exposed to body temperature or other elevated temperature, as discussed hereabove. Distal end 142 engages a scapula upon a phase transformation, as shown in FIG. 3B. Fixation wire 140 may be geometrically configured in various configurations as shown in FIG. 4A-H herebelow, such as to increase the frictional engagement of wire 140 with the scapula.

Prior to insertion of fixation wire 140, a hole is drilled into a scapula. In a typical embodiment, the hole has a diameter of approximately 0.04″. In some embodiments, the hole may be smaller than 0.04″. In other embodiments, the hole may be bigger than 0.04″. The phase change to wire 140 is used to initially fix wire 140 and glenoid body 103 to the scapula. Subsequent to inserting wire 140 into the scapula, bone in-growth will occur, thus further securing wire 140 in place.

FIGS. 4A-H are side views of various fixation wire configurations to be included within an implantable glenoid prosthesis. The configurations shown are achieved after wire 140 has been inserted, and a phase change initiated, as described hereabove. Prior to phase change, wire 140 may have a straight or other geometric profile. The varying geometries of fixation wire 140 after the phase change provide alternative configurations for increased strength and/or the frictional engagement between fixation wire 140 and a scapula into which it has been inserted. By increasing the strength and/or friction, sufficient stability to support glenoid body 103 is achieved prior to bone in-growth, and the longevity of the implant may be increased.

In one embodiment, a hole, hole 160 is drilled into a scapula. Hole 160 typically has a uniform diameter, as shown in FIGS. 4A-D. Hole 160 diameter may be approximately 0.04″ or another dimension, as described above. Alternatively, hole 160 may include radially extended distal portion 161, as shown in FIGS. 4E-H. Radially extended distal portion 161 may be created after a unidiameter hole is dined, such as with a tool configured to radially extend beyond an existing hole's diameter. Radially extended distal portion 161 may improve the fixation of wire 140 with a scapula by providing increased surface area with which wire 140 may engage and/or by creating a flange surface upon which a part of fixation wire 140 may apply a retaining force.

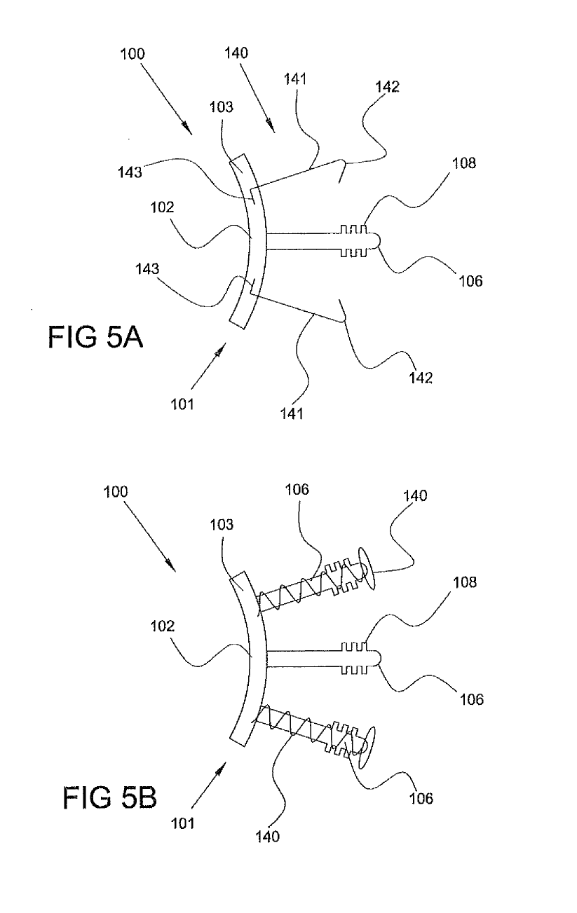

FIG. 5A is a side view of an implantable glenoid prosthesis including both peg fixation members and shape memory alloy fixation wires subsequent to a shaped memory phase change transformation of the wires. Glenoid prosthesis 100 includes glenoid member 101 having glenoid body 103 with glenoid surface 102. Glenoid prosthesis 100 further includes at least one glenoid fixation member, peg 106 having projections 108. The proximal end of peg 106 is attached to glenoid body 103. Projections 108 provide additional surface area for bone in-growth, thus strengthening and increasing the longevity of glenoid prosthesis 100.

Glenoid prosthesis 100 also includes a plurality of fixation wires 140. Wire 140 is preferably a shape memory alloy wire, for example, a Nitinol wire configured to undergo a phase change transformation when exposed to body temperature or another elevated temperature, as has been described hereabove. Proximal end 143 is secured to glenoid body 103, such as via a weld, a crimp and/or an adhesive joint. Distal end 142 engages a scapula upon the shaped memory phase transformation. Fixation wire 140 may be configured in the various geometries as described in FIGS. 4A-H hereabove. Prior to insertion of fixation wire 140, a hole is drilled into the scapula. In a typical embodiment, the hole has a diameter of approximately 0.04″. The phase change to wire 140 is used to initially fix wire 140 and glenoid body 103 to the scapula. Subsequent to inserting wire 140 into the scapula, bone in-growth will occur, thus further securing wire 140 in place.

As shown in FIG. 5B, fixation wire 140, in a helical construction, may be used in conjunction with peg 106 to provide both an initial securement, via phase change to wire 140, as well as additional surface area for bone in-growth and thereby strengthen glenoid prosthesis 100.

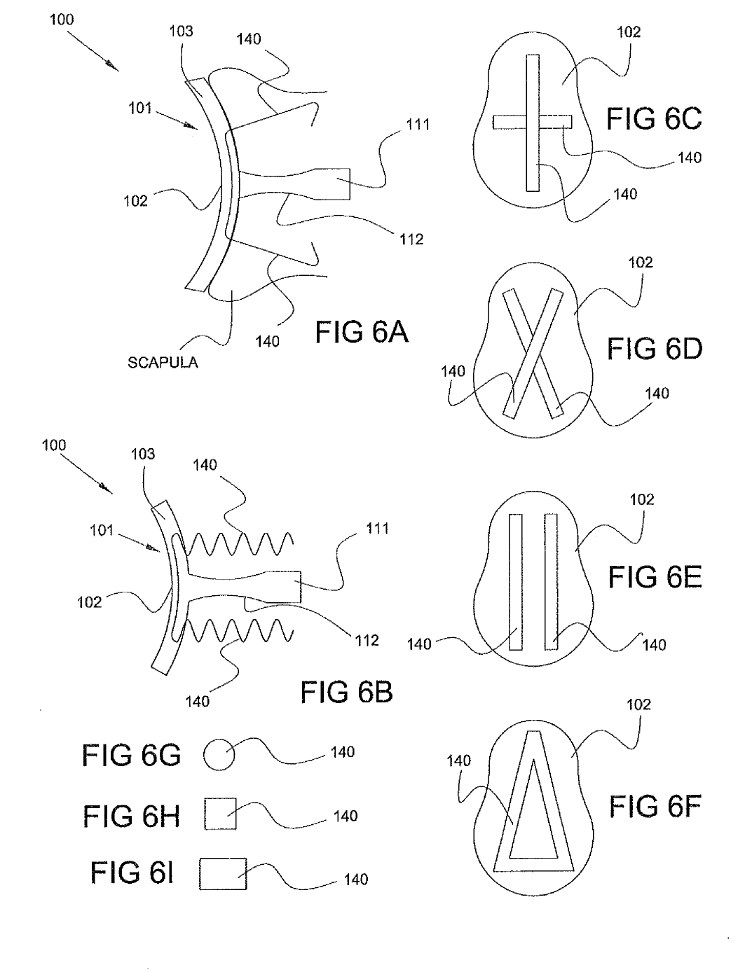

FIGS. 6A and 6B are side views of two configurations of an implantable glenoid prosthesis each including a glenoid fixation member and a plurality of shape memory alloy fixation wires, and each subsequent to a shaped memory material phase transformation. Glenoid prosthesis 100 includes glenoid member 101 having glenoid body 103 with glenoid surface 102. Glenoid prosthesis 100 further includes at least one fixation element, fin 111 having at least one recess 112. Fin 111 may be configured to flex, such as a Nitinol fin configured to be relatively elastic or otherwise resiliently biased to flex under load without plastic deformation. Glenoid prosthesis 100 also includes at least one fixation wire 140, shown as a single wire which passes through glenoid body 103, exiting the surface opposite joint surface 102 at two locations, such as to have each end of the single wire 140 inserted into one or more holes drilled into a scapula. Alternatively, two or more wires 140 may pass through or along a surface of glenoid body 103, each wire 140 having each end secured within the scapula. FIGS. 6C 6F are glenoid surface views of glenoid 100, showing varying patterns which two or more fixation wires 140 may take as they pass through or along a surface of glenoid body 103. These patterns change the displacement and vector orientation of forces that are transmitted from glenoid prosthesis 100 to the scapula, such as to limit possible loosening of glenoid prosthesis 100 over time. FIGS. 6G-6I are cross sectional views of fixation wire 140, showing varying geometries which have different flexural characteristics and may be configured to increase surface contact between fixation wire 140 and the scapula and/or the strength of the fixation. Fixation wires 140 may have a relatively constant cross-section, or a cross section that varies along its length.

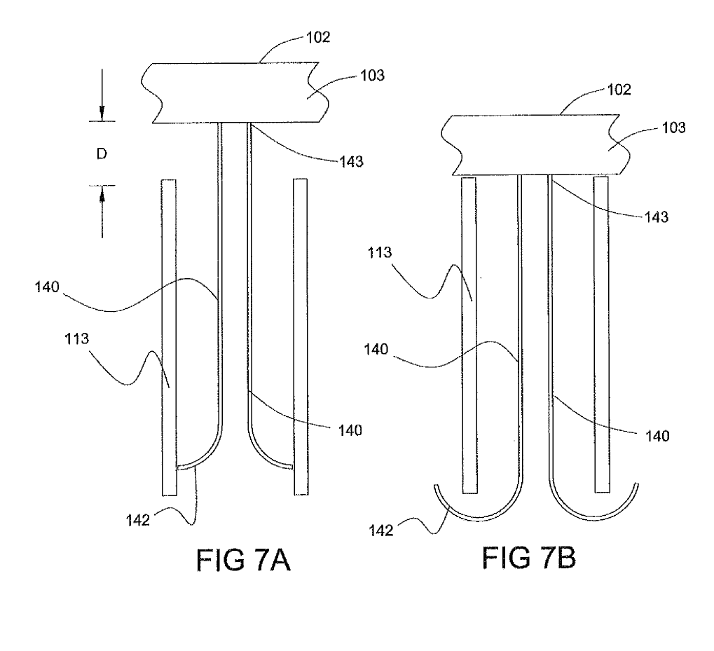

FIGS. 7A and 7B are side sectional views of an implantable glenoid prosthesis, shown prior to and after engagement with a scapula, respectively, and including a plurality of shape memory alloy fixation wires and a retaining tube. Fixation wire 140 is preferably a shape memory alloy wire, for example, a Nitinol wire configured to undergo a shaped memory phase transformation, as discussed hereabove. Proximal end 143 is secured to glenoid body 103, such as via a weld, a crimp and/or an adhesive joint. As glenoid joint surface 102 is moved in a direction such that distance D decreases, distal portion 142 of fixation wires 140 eventually exit the distal end of, and engage, retaining tube 113. Retaining tube 113 may comprise metal or plastic biocompatible materials, such as Nitinol. Tube 113 may be made of bioabsorbable materials, such as magnesium, and be configured to degrade over time.

Prior to insertion of retaining tube 113 and fixation wire 140, a hole, sized for insertion of tube 113, is drilled into a scapula. Tube 113 may be inserted prior to or concurrent with fixation wire 140. Wire 140 may include various geometric configurations, such as those described in reference to FIGS. 4A-H.

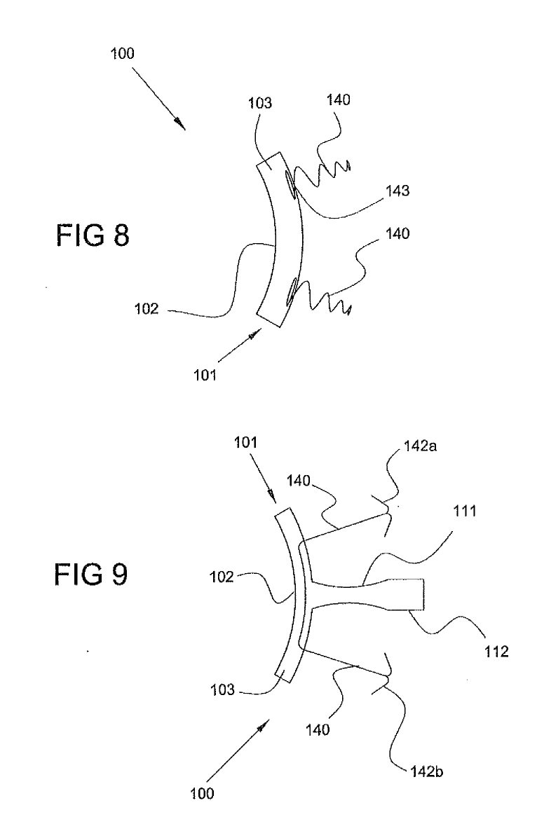

FIG. 8 is a side view of an implantable glenoid prosthesis including a plurality of shape memory alloy fixation wires in a coiled configuration subsequent to a shape memory phase transformation. Glenoid prosthesis 100 includes glenoid member 101 having glenoid body 103 with glenoid surface 102. Glenoid prosthesis 100 further includes at least one fixation wire 140 having a distal end, proximal end 143, and a body portion therebetween. Fixation wire 140 is preferably a shape memory alloy wire, for example, a Nitinol wire configured to undergo a shaped memory phase transformation, as discussed hereabove. Proximal end 143 is secured to glenoid body 103, such as via a weld, a crimp and/or an adhesive joint. Wire 140 engages a scapula upon a shaped memory phase transformation. In the illustrated embodiment, fixation wire 140 is in a coiled or “cork-screw” configuration. This configuration may increase the strength and/or the friction between fixation wire 140 and a scapula, such as an increase at the time of implantation and thereafter.

Prior to insertion of fixation wire 140, a hole is drilled into a scapula. In a typical embodiment, the hole has a diameter of approximately 0.04″. The phase change to wire 140 is used to initially fix wire 140 and glenoid body 103 to the scapula. Subsequent to inserting wire 140 into a scapula, bone in-growth will occur, thus further securing wire 140 in place.

FIG. 9 is a side view of an implantable glenoid prosthesis including a glenoid fixation member and a plurality of shape memory alloy fixation wires subsequent to a shaped memory phase transformation. Glenoid prosthesis 100 includes glenoid member 101 having glenoid body 103 with glenoid surface 102. Glenoid prosthesis 100 further includes at least one glenoid fixation member, fin 112 having at least one recess 111. Fin 112 is preferably manufactured of a flexible or semi-rigid material, such as to achieve a construction with similar properties of the scapula into which fin 112 is to be implanted. Fin 112 may be made of a metal or combination of metals, and have a thickness such that fin 112 can flex under normal load conditions. In one embodiment, fin 112 is made of Nitinol or includes one or more Nitinol portions. In another embodiment, fin 112 is made of a different metal or other material and sized to flex under normal load conditions, such as a material selected from the group consisting of: Nitinol; cobalt-chrome; titanium; stainless steel; tantalum; polyethylene; Delrin; silicon; nylon; and combinations thereof. The proximal end of fin 112 is attached to glenoid joint surface 102 of glenoid member 101. Recess 111 provides additional surface area for bone in-growth and thereby strengthens glenoid prosthesis 100. Fin 112 may have a similar construction to the fin of FIGS. 2A and 2B.

Glenoid prosthesis 100 also includes at least one fixation wire 140, shown as a single wire which passes through glenoid body 103, exiting the surface opposite joint surface 102 at two locations, such as to have each end of the single wire 140 inserted into one or more holes drilled into a scapula. Fixation wire 140 has a first end 142a and a second end 142b. Fixation wire 140 is preferably a shape memory alloy wire, for example, a Nitinol wire configured to undergo a shaped memory phase transformation, as discussed hereabove. After the phase transformation, ends 142a and 142b engage the scapula in which it has been inserted. In the illustrated embodiment, ends 142a and 142b each comprise a split end, with two filaments extending from a single shaft. The dual filament configuration may increase the strength and/or the frictional engagement between fixation wire 140 and the scapula. Prior to insertion of fixation wire 140, two holes are drilled into the scapula, one each for insertion of end 142a and 142b. In a typical embodiment, the hole has a diameter of approximately 0.04″. The phase change to wire 140 is used to initially fix wire 140 and glenoid body 103 to the scapula. Subsequent to inserting wire 140 into a scapula, bone in-growth will occur, thus further securing wire 140 in place.

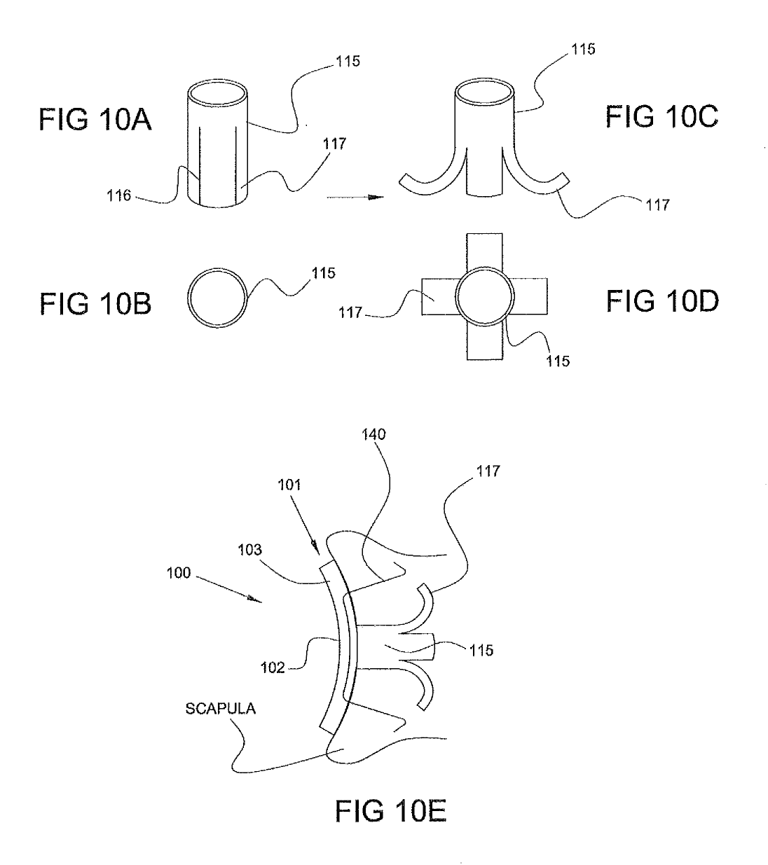

FIGS. 10A and 10B are side and end views, respectively, of a glenoid fixation member comprising a slit tube construction. Fixation member, tube 115 is configured to be attached or attachable to a glenoid body, as has been described hereabove and as is specifically described in reference to FIG. 10E herebelow. Fixation member 115 includes multiple slits 116 along a portion of its length, circumferentially separated, which define fixation portions 117. At least one fixation portion 117 comprises a shape memory alloy, such as Nitinol, shown in FIGS. 10A and 10B prior to a shaped memory phase transformation. FIGS. 10C and 10D are side and end views, respectively, of fixation member 115, with fixation portions 117 shown having transitioned into the curved and radially extended configuration. The transition is typically accomplished with exposure to an elevated temperature, such as an exposure to body temperature once implanted, as described hereabove. FIG. 10E shows glenoid prosthesis 100 including fixation member 115 of FIGS. 10A and 10B, with fixation portions 117 engaged with a scapula, in the curved and radially extended state (deployed condition). Glenoid prosthesis 100 also includes two fixation wires 140 as have been described in reference to multiple figures hereabove. Prior to implantation, a hole sized similar or slightly larger to the diameter of tube 115, is drilled into a scapula. Holes are drilled for fixation wires 140, such as two holes comprising a diameter of approximately 0.04″. Glenoid prosthesis 100 including tube 115 and wires 140, prior to phase transformation, is attached to the scapula by inserting tube 115 and wires 140 into the appropriate holes. Glenoid prosthesis 100, and the other glenoid prosthesis described throughout this application, may include a hole, notch or other recess drilling or cutting template such that the holes, notches or other recesses are properly aligned with the fixation elements of the particular glenoid prosthesis.

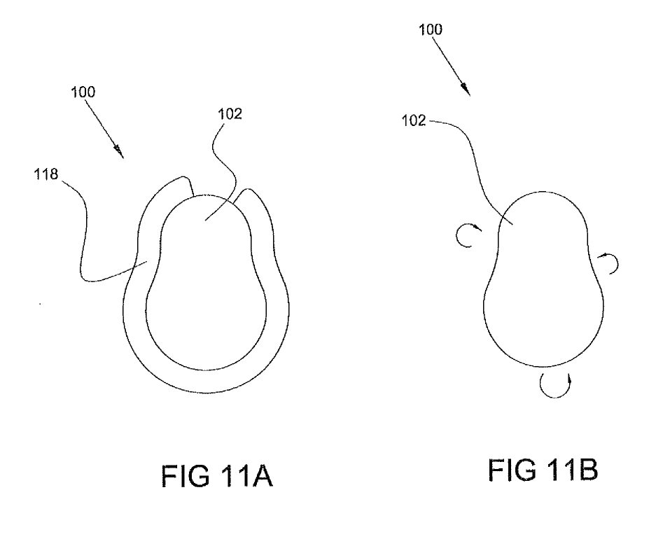

FIGS. 11A and 11B are undeployed and deployed views, respectively, of an implantable glenoid prosthesis including a foldable flange along its periphery. Glenoid prosthesis 100 includes glenoid joint surface 102 configured to rotatably interface with a natural or artificial humeral head. Surrounding glenoid joint surface 102 is a foldable edge, flange 118, configured as a glenoid fixation member of the present inventive concepts. As prosthesis 100 is placed proximate the patient's scapula, flange 118 is folded in the directions shown by arrows of FIG. 11B, such as to curve out and then back toward the scapular surface, similar to a “bottle-cap” attachment. Flange 118 may be made of a shape memory material, such as Nitinol, and all or part of the folding may be part of a phase change, such as a phase change that occurs during or after glenoid prosthesis 100 is implanted and flange 118 transitions to body temperature. Flange 118 may be configured to be manually folded, with any phase change transformation causing additional folding and thus applying additional retaining forces. Glenoid prosthesis 100 may include other glenoid fixation elements, such as fins, pegs, screws and the various glenoid fixation elements described throughout this application that are attached to and extend from the surface opposite surface 102.

Each of the embodiments of the glenoid prosthesis of the present inventive concepts includes one or more glenoid fixation members that are configured to prevent loosening of the glenoid prosthesis over time. Though not specifically shown, each embodiment may include combinations of two or more fixation members that are described singly in reference to the above drawings. For example, the flexible fin fixation element of FIGS. 2A and 2B, may be combined with the foldable flange design of FIGS. 11A and 11B, or with any of the wire fixation element designs described in reference to multiple drawings. Alternatively or additionally, each of the glenoid fixation members may be combined with standard screw, fin or other attachment elements common to artificial glenoid fixation to a scapula.

While the preferred embodiments of the devices and methods have been described in reference to the environment in which they were developed, they are merely illustrative of the principles of the inventive concepts. Modification or combinations of the above-described assemblies, other embodiments, configurations, and methods for carrying out the embodiments, and variations of aspects of the inventive concepts that are obvious to those of skill in the art are intended to be within the scope of the claims.

1-59. (canceled)

60. A method for implanting a glenoid prosthesis comprising:
implanting a glenoid body; and
attaching the glenoid body to a scapula via a glenoid fixation member;
wherein the glenoid fixation member is constructed and arranged to flex when a force is applied to the glenoid body.

61. The method of claim 60 wherein the glenoid fixation member comprises a shaped memory alloy material configured to undergo a phase change.

62. The method of claim 61 wherein the glenoid body attaches to the scapula upon the phase change.

63. The method of claim 62 wherein the phase change occurs when the shaped memory alloy material is heated.

64. The method of claim 63 wherein the phase change occurs when the shaped memory alloy material transitions to body temperature.

65. The method of claim 63 wherein the phase change occurs when a current is passed through the shaped memory alloy material.

66. The method of claim 63 wherein the phase change occurs when the shaped memory alloy material is heated via a heating device.

67. The method of claim 66 wherein the heating device comprises a heat gun.

68. The method of claim 61 wherein the shaped memory alloy material is constructed and arranged to pivot at least a portion of the glenoid fixation member.

69. The method of claim 61 wherein the shaped memory alloy material is constructed and arranged to mechanically engage the scapula.

70. The method of claim 60 wherein the glenoid fixation member comprises at least one in-growth element selected from the group consisting of: a hole; a projection; a flange; a notch; a recess; a groove; and combinations thereof and wherein the glenoid body attaches to the scapula via bone in-growth.

71. The method of claim 60 wherein the glenoid fixation member attaches the glenoid body to the scapula via at least one of: bone cement; at least one bone screw; or a press fit.

72. The method of claim 60 wherein the glenoid fixation member comprises a retaining tube having a proximal end and a distal end, and at least one wire wherein the at least one wire is advanced such that it engages the distal end of the retaining tube.

73. The method of claim 72 wherein the wire comprises a shaped memory alloy material constructed and arranged to undergo a phase change.

74. The method of claim 60 wherein the glenoid fixation member comprises a flange and wherein the glenoid body is attached to the scapula via folding the flange.

75. The method of claim 74 wherein the flange is manually folded.

76. The method of claim 74 wherein the flange comprises a shaped memory alloy material and wherein the flange is folded via a phase change of the shaped memory alloy material.

77. The method of claim 60 further comprising drilling at least one hole in the scapula.

78. The method of claim 77 wherein the at least one hole comprises a diameter of approximately 0.04″.

79. The method of claim 78 wherein the at least one hole comprises a radially extended distal portion.

80-175. (canceled)
